(19) United States
(12) Patent Application Publication
(10) Pub. No.: US 2023/0097382 A1
(43) Pub. Date: Mar. 30, 2023

(54) DEVICES AND METHODS FOR QUANTIFYING FATTY ACIDS

(71) Applicant: Nutrasource Pharmaceutical and Nutraceutical Services Inc., Guelph (CA)

(72) Inventors: William Rowe, Guelph (CA); Michael Barr, Guelph (CA); Theo Kapanadze, Guelph (CA)

(21) Appl. No.: 17/787,052

(22) PCT Filed: Dec. 18, 2020
(86) PCT No.: PCT/CA2020/051759

§ 371 Date: Jun. 17, 2022

Related U.S. Application Data
(60) Provisional application No. 62/951,321, filed on Dec. 20, 2019.

(51) Int. Cl. B01L 3/00; G01N 33/92; G01N 21/25

(52) U.S. Cl. CPC B01L 3/502715; G01N 33/92; G01N 21/25; B01L 2200/027; B01L 2200/0605; B01L 2200/10; B01L 2300/044; B01L 2300/0636; B01L 2300/0672; B01L 2300/0816; B01L 2400/0683; G01N 2021/6439

(57) ABSTRACT

Microfluidic devices and methods of quantifying fatty acids and/or specialized pro-resolving mediators and/or fatty acid metabolites present in a fluid sample on a microfluidic device are described herein. The methods include extracting fatty acid esters containing fatty acids from the fluid sample, combining the extracted fatty acid esters with a hydrolyzing agent to cleave the fatty acids from the extracted fatty acid esters and form free fatty acids, and quantifying the free fatty acids by performing a bioassay specific to the free fatty acids. Microfluidic devices and methods of quantifying fatty acid metabolites present in a fluid sample on a microfluidic device are also described herein.

### TECHNICAL FIELD

The embodiments disclosed herein relate to devices and methods for quantifying molecules in fluid samples, and more specifically to devices and methods for quantifying omega-3 fatty acids and/or fatty acid metabolites in fluid samples on microfluidic devices.

### BACKGROUND

The two major classes of polyunsaturated fatty acids are omega-3 fatty acids and omega-6 fatty acids. Like all fatty acids, omega-3 and omega-6 fatty acids consist of long chains of carbon atoms with a carboxyl group at one end of the chain and a methyl group at the other end. Omega-3 and omega-6 fatty acids are distinguished from saturated and monounsaturated fatty acids by the presence of two or more double bonds between carbons within the fatty acid chain. Omega-3 fatty acids have a carbon-carbon double bond located three carbons from the methyl end of the chain whereas omega-6 fatty acids have a carbon-carbon double bond located six carbons from the methyl end of the chain.

Omega-3 fatty acids are present in certain foods such as flaxseed and fish, as well as dietary supplements such as fish oil. Several different omega-3 fatty acids exist, but the majority of scientific research focuses on three specific omega-3 fatty acids: alpha-linolenic acid (ALA), eicosapentaenoic acid (EPA), and docosahexaenoic acid (DHA). ALA contains 18 carbon atoms, EPA contains 20 carbon atoms and DHA contains 22 carbon atoms. The molecular structure of DHA is shown in Formula 1, below:

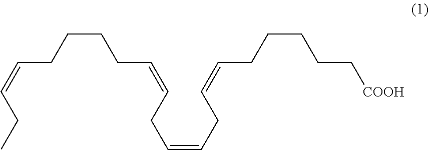 [SMILES: CC/C=C\CCCC/C=C\C/C=C\C/C=C\CCCCCC(=O)O]

Omega-3 fatty acids play an important role in the human diet and human physiology. Specifically, omega-3 fatty acids play important roles in the body as the components of phospholipids that form structures of cell membranes. DHA, in particular, is present in especially high concentrations in the retina, brain, and testis.

Further to this structural role, omega-3 fatty acids also provide energy for the body and are used to form eicosanoids, molecules that are important in cell signaling. It has also been reported that resolvins (e.g. RvD1), metabolic by-products of omega-3 fatty acids, are involved in inflammation resolution. Inflammation is linked to many chronic diseases such as but not limited to cancer and Alzheimer's.

Some studies suggest that low levels of omega-3 fatty acids in the body can be indicators of a risk of cancer, heart failure, cardiovascular disease and, in pregnant women, risk of premature birth. Omega-3 fatty acid levels have also been linked to a patient's risk of depression, arthritis pain, diabetes and weight loss. It is therefore important to be able to accurately measure and quantify levels of omega-3 fatty acids in the body.

Quantifying omega-3 fatty acid levels in the body is typically done by measuring fatty acid levels in plasma or serum phospholipids, or via analysis of erythrocyte (red blood cell) fatty acids. Plasma and serum fatty acid levels have traditionally been more difficult to quantify. Quantifying omega-3 fatty acid levels via analysis of erythrocyte fatty acids typically requires complicated laboratory analysis and can often take long periods of time to receive results.

Point-of-care diagnostic testing, defined as medical diagnostic testing at or near the point of care (i.e. at the time and place of patient care), offers convenience, expedited results and low costs to physicians and patients when compared to traditional laboratory-based diagnostic testing. At-home tests (i.e. tests directly available to a patient to perform themselves), offer the above-noted advantages of point-of-care diagnostic testing as well as privacy when compared to traditional laboratory-based diagnostic testing.

Accordingly, there is a need for methods for quantifying omega-3 fatty acid levels in a fluid that are implemented in a point of care device.

### SUMMARY

Methods of quantifying fatty acids present in a fluid sample on a microfluidic device are described herein. The methods include extracting phospholipids containing the fatty acids from the fluid sample; combining the extracted phospholipids with a hydrolyzing agent on the microfluidic device to cleave the fatty acids from the extracted phospholipids and form free fatty acids; and quantifying the free fatty acids by performing a bioassay specific to the free fatty acids on the microfluidic device.

Methods of quantifying a specialized pro-resolving mediator present in a fluid sample on a microfluidic device are also described herein. The methods include extracting fatty acid esters containing fatty acids from the fluid sample; combining the extracted fatty acid esters with a hydrolyzing agent on the microfluidic device to cleave fatty acids from the extracted fatty acid esters and form free fatty acids; converting the free fatty acids to the specialized pro-resolving mediator to be detected in a bioassay; and quantifying the specialized pro-resolving mediator by performing a bioassay specific to the specialized pro-resolving mediator on the microfluidic device.

Methods of quantifying a fatty acid metabolite present in a fluid sample on a microfluidic device. The methods include extracting fatty acid esters from the fluid sample; combining the extracted fatty acid esters with a hydrolyzing agent on the microfluidic device to cleave fatty acids from the extracted fatty acid esters; converting the free fatty acids to the fatty acid metabolite to be detected in a bioassay; and quantifying the fatty acid metabolite by performing a bioassay specific to the fatty acid metabolite on the microfluidic device.

In some embodiments, extracting the fatty acid esters from the fluid sample includes combining the fluid sample with an extraction agent stored on the microfluidic device.

In some embodiments, the extraction agent is an organic solvent.

In some embodiments, after combining the extracted fatty acid esters with the hydrolyzing agent on the microfluidic device to form the free fatty acids: converting the free fatty acids to a fatty acid metabolite to be detected in the bioassay; and quantifying the free fatty acids by detecting the fatty acid metabolite with an antibody unique to the fatty acid metabolite.

In some embodiments, the free fatty acids include DHA and the DHA is converted to RvD1.

In some embodiments, the DHA is converted to RvD1 by combining the DHA with an enzyme.

In some embodiments, the enzyme is selected from a group consisting of: 5-lipoxgenase (5-LOX), soybean lipoxgenase, 12-lipoxgenase (12-LOX), 15-lipoxgenase (15-LOX), human ALOX15-2, cyclooxygenase-1 (COX-1), cyclooxygenase-2 (COX-2), Cy P450, a combination of 15-LOX and 5-LOX, a combination of 12-LOX and 15-LOX and a combination of cyclooxygenase-2 (COX-2) and acetylsalicylic acid (ASA).

In some embodiments, the method further comprises, after combining the extracted fatty acid esters with the hydrolyzing agent on the microfluidic device to form the free fatty acids: conjugating the free fatty acids to be detected in the bioassay; and quantifying the free fatty acids by detecting the conjugated fatty acids with an antibody unique to the conjugated fatty acid.

In some embodiments, the hydrolyzing agent is a chemical hydrolyzing agent.

In some embodiments, the chemical hydrolyzing agent comprises one or more of the following: potassium hydroxide (KOH), sodium hydroxide (NaOH), deacylation with lithium hydroxide in chloroform-methanol, and/or hydrochloric acid (HCl)

In some embodiments, the hydrolyzing agent is an enzymatic hydrolyzing agent.

In some embodiments, the enzymatic hydrolyzing agent is selected from a group consisting of: lipases, phospholipases, phospholipases A (PLA), phospholipase A2 (PLA2s), Ca2+-independent phospholipase A2 (iPLA2 β), cytosolic phospholipases A2 (cPLA2s), lipoprotein-associated phospholipases A2 (Ip-PLAs2), secreted phospholipases (sPLA2s), phospholipases D, Burkholderia cepacia (BC) lipase Chromobacterium viscosum lipase, Lipase A Candida antarctica, Rhizopus oryzae (RO) lipase, pancreatin lipase, and a combination of two or more of any of the above.

In some embodiments, the methods further include, prior to combining the fluid sample with an organic solvent, normalizing a volume of the fluid sample.

In some embodiments, after combining the fluid sample with an extraction agent, normalizing a sample volume containing extracted fatty acid esters.

In some embodiments, the methods include filtering the fluid sample into red blood cells and plasma prior to normalizing a volume of the fluid sample.

In some embodiments, the methods include filtering the fluid sample into red blood cells and plasma after normalizing a volume of the fluid sample.

In some embodiments, the bioassay generates a colorimetric response to quantify the free fatty acids, the specialized pro-resolving mediator or the fatty acid metabolite.

In some embodiments, the bioassay generates an electrochemical response to quantify the free fatty acids, the specialized pro-resolving mediator or the fatty acid metabolite.

In some embodiments, the bioassay generates a response detectable by a cellular phone spectrophotometer.

According to another broad aspect, a microfluidic device for quantifying fatty acids in a fluid sample is described herein. The microfluidic device includes: a sample preparation module configured to receive the fluid sample, the sample preparation module being configured to: extract fatty acid esters containing the one or more fatty acids from the fluid sample; and retain a hydrolyzing agent to cleave the one or more fatty acids from the extracted fatty acid esters and form free fatty acids; and a detecting module fluidly coupled to the sample preparation module and configured to receive the free fatty acids from the sample preparation module; wherein one of the sample preparation module and the detecting module retains an assay agent to be bound to the free fatty acids to quantify the free fatty acids in the fluid sample.

According to another broad aspect, a microfluidic device for quantifying a specialized pro-resolving mediator in a fluid sample is described herein. The microfluidic device includes: a sample preparation module configured to receive the fluid sample, the sample preparation module having a reservoir configured to: extract fatty acid esters containing one or more fatty acids from the fluid sample; and retain a hydrolyzing agent to cleave the one or more fatty acids from the extracted fatty acid esters and form free fatty acids; and a detecting module fluidly coupled to the sample preparation module and configured to receive the free fatty acids from the sample preparation module; wherein one of the sample preparation module and the detecting module retains an assay agent to be bound to the free fatty acids to quantify the specialized pro-resolving mediator in the fluid sample.

According to another broad aspect, a microfluidic device for quantifying a fatty acid metabolite in a fluid sample is described herein. The microfluidic device includes a sample preparation module configured to receive the fluid sample, the sample preparation module having a reservoir configured to: extract fatty acid esters containing one or more fatty acids from the fluid sample; and retain a hydrolyzing agent to cleave the one or more fatty acids from the extracted fatty acid esters and form free fatty acids; and a detecting module fluidly coupled to the sample preparation module and configured to receive the free fatty acids from the sample preparation module; wherein one of the sample preparation module and the detecting module retains an assay agent to be bound to the free fatty acids to quantify the fatty acid metabolite in the fluid sample.

In some embodiments, the sample preparation module is further configured to retain an extraction agent for extracting the fatty acid esters from the fluid sample.

In some embodiments, the extraction agent is an organic solvent.

In some embodiments, the sample preparation module comprises a blood filtration unit configured to separate whole blood into red blood cells (RBCs) and plasma.

In some embodiments, the sample preparation module comprises a normalization module configured to normalize a volume of the fluid sample.

In some embodiments, the sample preparation module comprises a reaction chamber configure to receive the fluid sample, the reaction chamber being fluidly coupled to the reservoir and configured to receive one or more of the organic solvent and the hydrolyzing agent from the reservoir to combine with the fluid sample.

In some embodiments, the reservoir is configured to receive the fluid sample to combine one or more of the organic solvent and the hydrolyzing agent with the fluid sample.

In some embodiments, the sample preparation module is configured to retain an agent configured to: convert the free fatty acids to a fatty acid metabolite to be detected in a bioassay.

In some embodiments, the detection module is configured to retain an agent configured to: convert the free fatty acids to a fatty acid metabolite to be detected in a bioassay.

In some embodiments, the free fatty acids include DHA and the DHA is converted to RvD1.

In some embodiments, the DHA is converted to RvD1 by combining the DHA with an enzyme.

In some embodiments, the enzyme is selected from a group consisting of: COX-1, 5-lipoxgenase (5-LOX), 12-lipoxgenase (12-LOX), 15-lipoxgenase (15-LOX), a combination of COX-2 and 5-LOX, cyclooxygenase-2 (COX-2), a combination of 15-LOX and 5-LOX, a combination of 12-LOX and 15-LOX and a combination of cyclooxygenase-2 (COX-2) and acetylsalicylic acid (ASA).

In some embodiments, the hydrolyzing agent is a chemical hydrolyzing agent.

In some embodiments, the chemical hydrolyzing agent comprises one or more of the following: potassium hydroxide (KOH), sodium hydroxide (NaOH), deacylation with lithium hydroxide in chloroform-methanol, and/or hydrochloric acid (HCl).

In some embodiments, the hydrolyzing agent is an enzymatic hydrolyzing agent.

In some embodiments, the enzymatic hydrolyzing agent is selected from a group consisting of: lipases, phospholipase, phospholipases A (PLA), phospholipase A2 (PLA2s), cytosolic phospholipases A2 (cPLA2s), iPLA2, Ca2+-independent phospholipase A2 (iPLA2 β), lipoprotein-associated phospholipases A2 (Ip-PLAs2), secreted phospholipases (sPLA2s), phospholipases D, Burkholderia cepacia (BC) lipase, Chromobacterium viscosum lipase, Lipase A Candida antarctica, Rhizopus oryzae (RO) lipase, pancreatin lipase, and a combination of two or more of any of the above.

In some embodiments, the normalization module is a microchannel configured to normalize a volume of the fluid sample before the fluid sample enters the sample preparation module.

In some embodiments, the microfluidic device is a lateral flow device.

In some embodiments, a user adding the fluid sample to the microfluidic device may expect to receive an indicator of the quantity of the fatty acids or SPMs or fatty acid metabolites in about two hours or less.

In some embodiments, a period of time between a user adding the fluid sample to the microfluidic device and receiving an indicator of the quantity of the free fatty acids or SPMs from the microfluidic device is in a range of less than about 1 minute to about 30 minutes.

In some embodiments, the period of time between the user adding the fluid sample to the microfluidic device and receiving the indicator of the quantity of the free fatty acids or SPMs from the microfluidic device is about 20 minutes.

In some embodiments, the fatty acids are omega-3 fatty acids.

In some embodiments, the fatty acids are omega-6 fatty acids.

In some embodiments, the fatty acid metabolite includes one or more of: lipoxins (Lx), resolvins (Rv), protectins (PD), neuroprotectins (NP), isofurans, isoprostanes, maresins (MaR), SPM intermediates (eg. 14-HDHA, 17-HDHA, 18-HEPE and endocannabinoids).

In some embodiments, the fatty acids are omega-3 fatty acids.

In some embodiments, the fatty acids are omega-6 fatty acids.

In some embodiments, the fatty acid metabolite includes one or more of: lipoxins (Lx), resolvins (Rv), protectins (PD), neuroprotectins (NP), isofurans, isoprostanes, SPM intermediates (eg. 14-HDHA, 17-HDHA, 18-HEPE) and maresins (MaR).

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a perspective view of a microfluidic device for quantifying omega-3 fatty acids, according to one embodiment;

FIGS. 2A-2D show top, side, first perspective and second perspective views, respectively, of a sample collection area of a microfluidic device for quantifying omega-3 fatty acids, according to one embodiment;

FIGS. 3A-C show images of fluid samples placed on various materials to determine the surface properties (e.g. hydrophilicity and hydrophobicity) of the materials;

FIGS. 4A and 4B show images demonstrating filling the microchannel of FIG. 1;

FIG. 4C shows an image demonstrating removing residual fluid from a sample collection area of the device of FIG. 1;

FIG. 4D shows a filled microchannel of the device of FIG. 1;

FIG. 4E shows tilting the device of FIG. 1 to drive the fluid across the hydrophobic portion;

FIGS. 5A and 5B show side and perspective views, respectively, of a blister for retaining fluids in a microfluidic device for quantifying omega-3 fatty acids, according to one embodiment; and

FIG. 6 shows a flowchart of a method of quantifying omega-3 fatty acids in a fluid sample.

The skilled person in the art will understand that the drawings, further described below, are for illustration purposes only. The drawings are not intended to limit the scope of the applicant's teachings in any way. Also, it will be appreciated that for simplicity and clarity of illustration, elements shown in the figures have not necessarily been drawn to scale. For example, the dimensions of some of the elements may be exaggerated relative to other elements for clarity. Further aspects and features of the example embodiments described herein will appear from the following description taken together with the accompanying drawings.

### DETAILED DESCRIPTION

Various devices and methods will be described below to provide an example of each claimed embodiment. No embodiment described below limits any claimed embodiment and any claimed embodiment may cover devices or methods that differ from those described below. The claimed embodiments are not limited to devices or methods having all of the features of any one device or method described below or to features common to multiple or all of the devices or methods described below.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

Generally, devices and methods for quantifying fatty acids and/or specialized pro-resolving mediators (SPMs) and/or other fatty acid metabolites are described herein. SPMs are have been shown to be good biomarkers for the presence of fatty acids because SPMs are fatty acid metabolites of specific parent fatty acids. For example, the SPM resolving D1 (RVD1), a D-series resolvin, is a fatty acid metabolite of the parent omega-3 fatty acid DHA only. Therefore, if any RVD1 is measured in a sample, it is known that at some point the RVD1 originated as the parent omega-3, DHA.

SPMs are a superior target for ligand binding assays because of the presence of hydroxyl group(s) that are added to the parent fatty acid when the parent fatty acid is enzymatically converted to an SPM. The metabolism of fatty acids to metabolite SPMs involves the addition of hydroxyl groups to the carbon tail of the fatty acid. The addition of hydroxyl groups to the carbon tail of fatty acids makes the molecules more stereospecific. That is, SPMs are more likely to have a ridged and unchanging 3D structure than parent fatty acid molecules whose shape and structure rotates, folds and changes. It has been shown to be very difficult to develop a specific and selective binding mechanism against and a small, flexible molecule. However, there is greater confidence in developing binding mechanisms (e.g. antibody, aptamer, etc.) against a stereospecific molecule such as an SPM.

It should be noted that the systems, methods and devices described herein can be used to measure amounts of physiologically free fatty acids, SPMs (bound or unbound), or other fatty acid metabolites (bound or unbound) (e.g. direct detection via blood to bioassay (i.e. no conversion chemistry required)) or amounts of total fatty acids via chemistry that generates free fatty acids. Herein, the term “free fatty acids” refers to non-bound fatty acids that are bioactive whereas the term “total fatty acids” refers to both non-bound fatty acids and bound fatty acids.

The devices described herein are generally referred to as microfluidic devices and/or lab-on-a-chip devices that can be used in non-laboratory settings and simplify the analytical process of quantifying fatty acids and/or fatty acid metabolites in the body. Herein, the term “microfluidic device” generally refers to a device having one or more fluid passages, chambers or conduits which have at least one internal cross-sectional dimension, e.g., depth width, length, diameter, etc., that is less than 5000 μm, and typically between 0.1 μm and 200 μm. In some embodiments, the fatty acids are omega-3 fatty acids or omega-6 fatty acids.

In some embodiments, the devices described herein can also be described as point-of-care devices. Herein, the term “point-of-care device” generally refers to a device that is used at or near the time and place of patient care.

In some embodiments, the devices described herein can also be described as lateral flow devices. Herein, the term “lateral flow device” generally refers to a device that include one or more fluid passages, chambers or conduits that spontaneously drive a fluid across the device (e.g. by capillary force).

In some embodiments, the methods of quantifying fatty acids and/or fatty acid metabolites in a fluid sample are performed on or with one or more microfluidic devices. In some embodiments, a user adding a fluid sample to the microfluidic device may expect to receive a response from the device, such as but not limited to a color change of a component on the device and/or an electronic readout based on cantilever biosensors, for example, in about one hour. In some embodiments, the period of time between a user adding a fluid to the device and the user receiving a response from the device can be in a range of less than one minute to about 30 minutes. In some embodiments, the period of time between a user adding a fluid to the device and the user receiving a response from the device is about 20 minutes. In some embodiments, the period of time between a user adding a fluid to the device and the user receiving a response from the device is less than about 20 minutes.

It should be noted that although the examples and embodiments described herein refer to the sample provided to the microfluidic devices and the sample used in the methods described herein is a blood sample, other fluid samples can be used in the devices and methods herein to measure a total amount of fatty acids, free fatty acids, SPMs or other fatty acid metabolites. The processes described here may be used to analyze a fatty acid composition from any biological sample containing fatty acids or derivatives thereof. For instance, the biological sample can be a blood component such as whole blood, plasma, serum, red blood cells, platelets, white blood cells, cholesterol esters, triglycerides, free fatty acids, plasma phospholipids, or mixtures thereof. The fatty acid, SPM or fatty acid metabolite to be analyzed may exist as various forms in the biological sample, such as triglycerides, diglycerides, monoglycerides, sterol esters, phosphatidyl ethanolamines, phosphatidyl cholines, free fatty acids, etc. Herein, the term “fatty acid esters” is used to refer to a source of fatty acids, including but not limited to triglycerides, phospholipids, and cholesteryl esters. Fatty acids are usually not found in organisms in their standalone form, but instead exist as three main classes of esters: triglycerides, phospholipids, and cholesteryl esters. In any of these forms, fatty acids are both important dietary sources of fuel for animals and they are important structural components for cells.

Quantification of fatty acids and/or fatty acid metabolites in the fluid sample on the microfluidic device can be by one of many potential quantification mechanisms (described further below). For instance, quantification of the fatty acids and/or fatty acid metabolites in the fluid sample may be based on but not limited to the generation of a detectable signal. The detectable signal can be in response to, for example, a change in electrical resistivity/electrochemical within the fluid sample, an electrical response that changes when a binding event occurs (e.g. when DHA binds), when a resistance of a material changes (based on change of material structure or production of chemical that changes electrical properties), microelectrodes detect change in conductivity of the fluid sample, etc.), chemiluminescence, magnetic stripe reader, electrochemical detection, or an optical detection method (e.g. a colorimetric response (colour change) such as is produced by an enzyme-linked immunosorbent assay (ELISA), or fluorescence measurement), or the like.

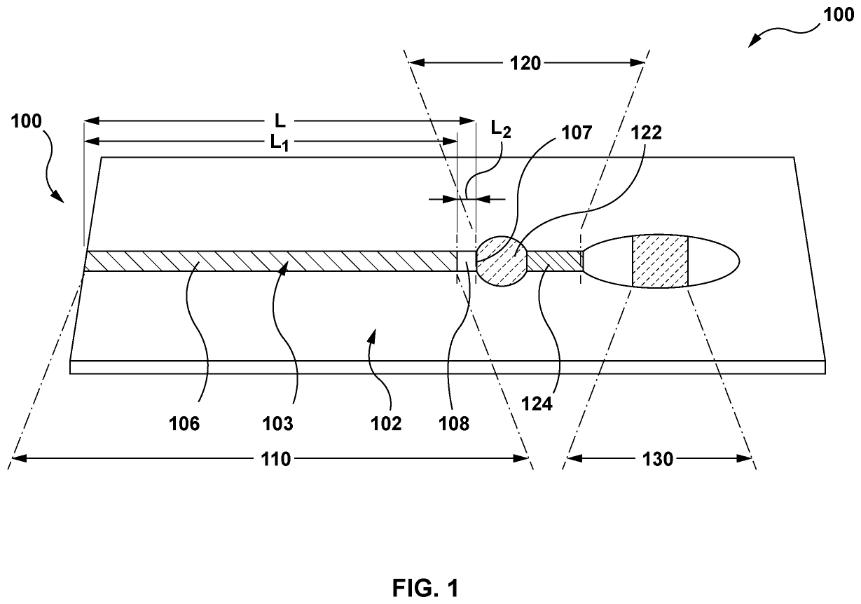

Referring now to FIG. 1, illustrated therein is a microfluidic device 100 for quantifying fatty acids such as but not limited to omega-3 fatty acids or omega-6 fatty acids, and/or fatty acid metabolites in a fluid sample, according to one embodiment. The fluid sample may be any fluid sample expected to contain fatty acids and/or the fatty acid metabolite(s) of interest.

For instance, microfluidic device 100 may be configured to quantify fatty acid levels and/or fatty acid metabolite(s) levels from whole blood. In some embodiments, microfluidic device 100 may be configured to separate whole blood into red blood cells (RBCs) and plasma prior to quantifying omega-3 fatty acid levels and/or fatty acid metabolite(s) levels. Microfluidic device 100 may then quantify omega-3 fatty acid levels and/or fatty acid metabolite(s) levels from blood serum (e.g. blood plasma without clotting factor (i.e. fibrinogens)).

In other embodiments, microfluidic device 100 may be configured to quantify fatty acids levels and/or fatty acid metabolite(s) levels in fluid samples other than whole blood. For instance, microfluidic device 100 may be configured to quantify omega-3 fatty acids and/or fatty acid metabolite(s) levels in fluid samples such as but not limited to saliva, breast milk and/or semen.

Generally, microfluidic devices described herein include two modules: a sample preparation module 120 and a detection module 130. In some embodiments, the sample preparation module 120 and the detection module 130 can be combined into a single unit (as described further below). In some embodiments, microfluidic devices described herein may include more than two modules. For instance, microfluidic devices described herein may include a dosage unit (or sample normalization) module 110 for normalizing a volume of the fluid sample prior to the fluid sample entering the sample preparation module 120. In the embodiment shown in FIG. 1, microfluidic device 100 includes a dosage unit module 110. Further, in some embodiments. Further, the microfluidic devices described herein may include submodules of the sample preparation module 120 and/or the detection module 130. These submodules may be used to perform chemistry on the fluid sample in various steps.

Microfluidic device 100 shown in FIG. 1 includes a substrate 102 in which at least one microchannel 104 is formed, such as but not limited to by a microfabrication process. Microfluidic device 100 may be made from one or more materials. For instance, glass object slides, which are commonly used in lab environments, may be used as a substrate 102. Glass object slides are generally inexpensive and are commonly used in various other analytical devices. Further, glass object slides generally have a hydrophilic surface treatment to enhance the spreading of fluids across at least one surface. To form microchannel 104 in a glass object slide, the glass slide may undergo a microfabrication process such as but not limited to an etching process.

In another example, pressure sensitive adhesive tapes (PSA) may also be used to form microchannel 104 of microfluidic device 100. Examples of specific PSAs that may be used include but are not limited to ARcare 92712® (Adhesive Research), ARcare 90445® (Adhesive Research), ARcare 90106® (Adhesive Research) and ARseal® (Adhesive Research).

In yet another example, polydimethylsiloxane (PDMS) may be used to form substrate 102 and microchannel 104, either alone or by being bonded together with one or more pieces of PDMS and/or one or more glass structures. In this example, the PDMS forming substrate 102 may undergo plasma activation to change one or more surface properties of the PDMS such as from being hydrophobic to being hydrophilic. Plasma activation may encourage the fluid to flow across a surface of the PDMS. Plasma treatment may also be used to have different surface renderings (e.g. both hydrophilic and hydrophobic portions) within one material surface in microfluidic device 100, for instance to provide a degree of control to the flow of a fluid across the material surface.

In some embodiments, substrate 102 may also be made of an absorptive membrane material and the fluid sample, after being placed on a top surface of the substrate 102, may be absorbed into the substrate 102 and travel laterally through the substrate 102.

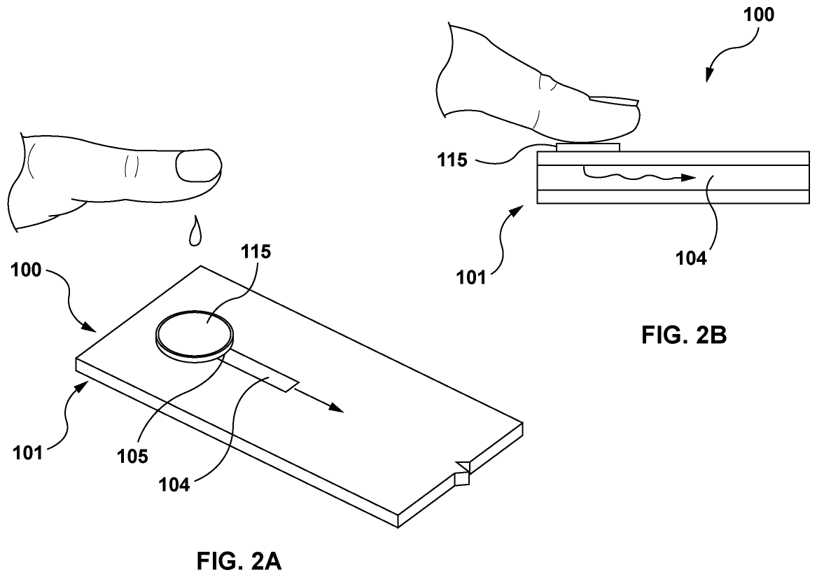

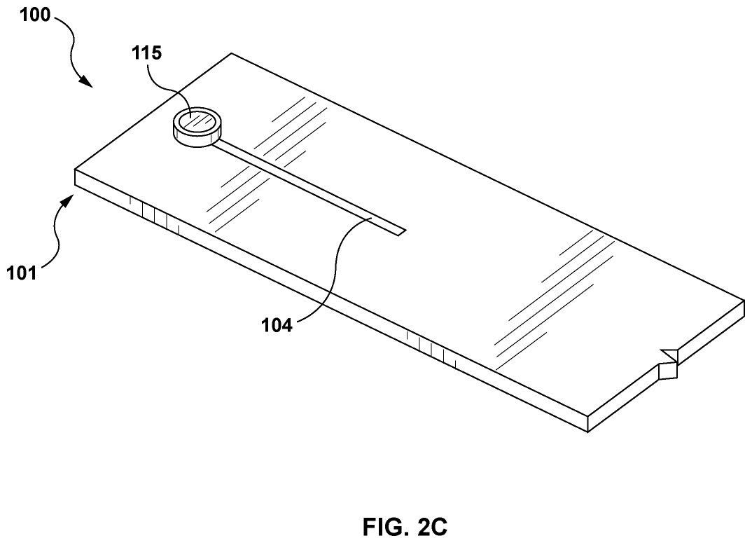

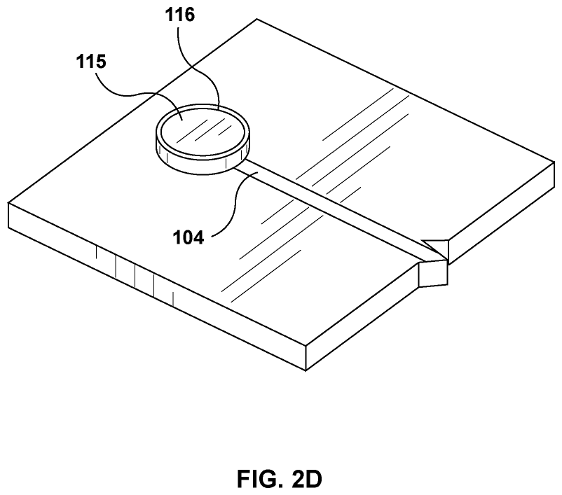

In some embodiments, the dosage unit module 110 includes a sample collection area 115, see FIGS. 2A to 2C, positioned at a first end 101 of the microfluidic device 100. The sample collection area 115 is configured to receive the fluid sample and to provide the fluid sample to the dosage unit 110 (i.e. microchannel 104). The fluid sample may be received at the sample collection area 115 in any appropriate manner, such as but not limited to being received directly from a syringe. In other embodiments, such as but not limited to the embodiment shown in FIGS. 2A and 2B, the sample collection area 115 may be configured to puncture a user's finger to receive the fluid sample directly from the user.

In some embodiments, the sample collection area 115 may be configured to perform blood separation by surface application of blood on a membrane (e.g. bound glass fibre or asymmetric polysulfone). In some embodiments, it may be necessary to pre-treat a surface of the sample collection area 115 with a substance to inhibit clotting in the sample collection area 115. Commonly used compounds appropriate to inhibit clotting include but are not limited to ethylenediaminetetraacetic acid (EDTA) and heparin. It should be understood that the substance to inhibit clotting in the sample collection area 115 should not react with DHA and/or EPA in the fluid sample. FIG. 2D shows one example embodiment where a membrane 116 surrounds a sample collection area 115. One or more surfaces of the membrane 116 may be pre-treated with an anti-coagulant to inhibit clotting of the fluid sample.

In embodiments where there is a concern that DHA may oxidize on the sample collection area 115, for instance in embodiments where the fluid sample is temporarily stored prior to undergoing a bioassay, one or more antioxidants may be included in the sample collection area 115 to inhibit oxidation of DHA. For example, butylated hydroxytoluene (BHT) or the like maybe used to inhibit DHA oxidation.

The sample collection area 115 is coupled to microchannel 104 at an inlet 105 of the microchannel 104. Microchannel 104 provides fluidic passage of the fluid sample between inlet 105 and an outlet 107. For instance, in the embodiment shown in FIG. 1, microchannel 104 is configured to draw fluid into the microchannel 104 from the sample collection area 115 and along the microchannel 104 towards the outlet 107. In at least one embodiment, microchannel 104 has a hydrophilic portion 106 having a length L1 and fluid is drawn into the microchannel 104 from the sample collection area 115 by capillary force.

Microchannel 104 is one embodiment of a structure to normalize a volume of the fluid sample prior to the fluid sample entering sample preparation module 120 (i.e. provide a defined volume of the fluid sample to the sample preparation module 120). Fluid sample normalization is important to accurately quantify the concentration of an analyte in a fluid sample. For instance, fluid normalization is important so that the quantification response of the following assay (e.g. optical, colorimetric, or electrochemical assay) can be accurately compared to a calibration scale.

In the embodiment shown in FIG. 1, normalization of a volume of the fluid sample is achieve in microchannel 104. Microchannel 104 has a hydrophobic portion 108 (see FIG. 1) having a length L2 that neighbor's hydrophilic portion 106. The hydrophilic portion 106 and the hydrophobic portion 108 together extend between the sample collection area (not shown) and the sample preparation module 120. As fluid enters the inlet 105 of the microchannel 104, the fluid is drawn along the microchannel 104 until the fluid reaches a hydrophobic portion 108 of the microchannel 104. Once at least a portion of the fluid reaches the hydrophobic portion 108, the fluid stops traveling along the microchannel 104. When the fluid sample fills the hydrophilic portion 106 of the microchannel 104, excess fluid can be removed from the sample collection area and the fluid remaining in the hydrophilic portion 106 of the microchannel 104 can be referred to as a defined volume of the fluid sample (i.e. the volume of fluid that fills the hydrophilic portion 106 of the microchannel 104) or a normalized volume of fluid.

Microchannel 104 is shown in the figures as having a generally straight line form along the length L between inlet 105 and outlet 107, however, it should be understood that microchannel 104 may have a different size and/or shape.

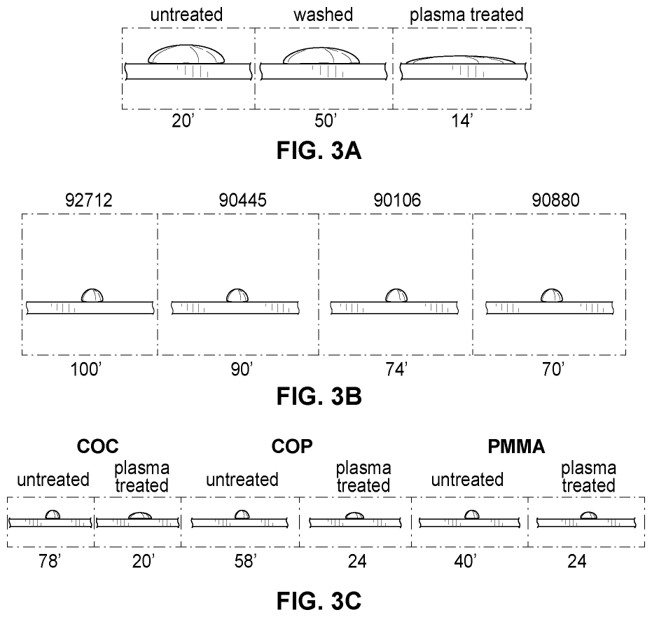

Turning to FIGS. 3A-3C, illustrated therein are images of fluid samples placed on various materials to determine the surface properties (e.g. hydrophilicity and hydrophobicity) of the materials for use in microfluidic device 100. In these experiments, the contact angles of 10 μl drops of fluid on three different PDMS samples with different surface treatments (untreated, washed and plasma treated) (FIG. 3A), four different adhesive tapes (FIG. 3B), and three different polymers (COC, COP and PMMA), each having two different surface treatments (untreated and plasma treated) (FIG. 3C) were measured. In these experiments, the fluid samples were blood that had been treated with EDTA to prevent clotting within the sample.

As shown in FIGS. 3A-3C, the PDMS, the different adhesive tapes and the different polymers show variations in a contact angle when comparing untreated and treated (plasma) surfaces. When these materials are used in the microfluidic device 100, the contact angle differences have an impact on the capillary forces within microchannel 104 and a change from a hydrophilic surface to a hydrophobic surface can be used as a flow barrier to normalize a volume of the fluid sample.

In some embodiments, a filter (not shown) can be positioned between the sample collection area 115 and microchannel 104 to filter debris and/or other impurities from the fluid sample prior to the fluid sample entering the microchannel 104. In some embodiments, the filter may be positioned before the sample normalization module 110. In these embodiments, the fluid sample being normalized may be blood plasma and the filter may receive a whole blood sample and provide blood plasma to the sample normalization module 110. In other embodiments, the filter may be positioned after the sample normalization module 110 and before the sample preparation module 120. In these embodiments, the fluid sample being normalized may be whole blood the filter may provide blood plasma directly to the sample preparation module 120.

For instance, in some embodiments where the fluid sample is a blood sample (i.e. whole blood), red blood cells (RBCs) in the whole blood can be separated from plasma (i.e. the liquid portion of the blood) in the whole blood by the filter. Once the RBCs have been removed from the plasma, the plasma is generally a cleaner solution with less biological debris from which the fatty acids can be more easily extracted and more accurately captured (as described below). Filtering is a well-established method to separate RBCs from whole blood to generate plasma and filters are commercially available. For instance, commercially available filters that may be used include but are not limited to the LF1 Glass Fiber Filter (GE Healthcare) and the Vivid Plasma Separation GF/GX/GR membrane (Pall Corporation).

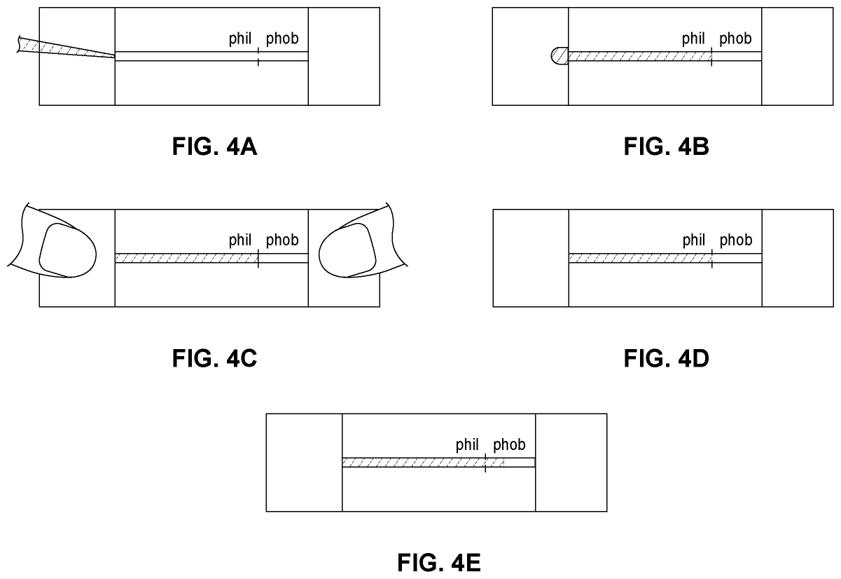

Once the microchannel 104 has been filled with fluid and any excess fluid has been removed from the sample collection area, or is otherwise inhibited from entering the microchannel 104, in some embodiments, a mechanical force, or another type of force (e.g. gravitational), can be applied to the microfluidic device 100 to drive the fluid sample within the microchannel 104 from the hydrophilic portion 106 through the hydrophobic portion 108 and into the sample preparation module 120. For instance, in some embodiments the microfluidic device 100 can be tilted such that the fluid sample contained in the hydrophilic portion 106 passes through the hydrophobic portion 108 and into the sample preparation module 120. An example of this is shown in FIG. 4, where FIGS. 4A and 4B show filing of the microchannel 104, FIG. 4C shows removing residual fluid, FIG. 4D shows a filled microchannel 104 prior to tilting and FIG. 4E shows tilting the microfluidic device 100 to drive the fluid across the hydrophobic portion 108 to be further processed.

It should be understood that fluid normalization may also occur using other suitable means. For instance, without limiting the foregoing, the normalization of the plasma volume may be controlled via the maximum soaking capacity of substrate 102 (e.g. a membrane). In this example, substrate 102 may comprise one ore more membranes. When substrate 102 comprises more than one membrane, the membranes may be laterally positioned relative to each other and cascading downwardly from a sample collection area 115 (e.g. a sample pad) towards an absorbent pad. As the fluid sample is absorbed by the sample pad it may travel laterally through one or more other membranes (e.g. a conjugate pad, a detection membrane, etc.) towards an absorbent pad. As the fluid sample is absorbed by the absorbent pad, once a soaking capacity of the absorbent pad is reached, lateral flow of the fluid into the absorbent pad will stop and the pad adjacent to the absorbent pad will fill with fluid. This process repeats until each of the membranes comprising the substrate 102 reaches their soaking capacity and the lateral flow of the fluid stops. Excess fluid may then dry on an upper surface of the sample collection area 115. As a result, the fluid volume passing through the detection area, for example, is independent from the added blood volume.

In the embodiment shown in FIG. 1, the sample preparation module 120 and the detection module 130 are shown as two separate units. However, it should be understood that in some embodiments the sample preparation module 120 and the detection module 130 can be combined into a single unit to receive the fluid sample and one or more reservoirs for retaining reactants (e.g. such as but not limited to an organic solvent and/or a hydrolyzing agent, as described below). In some embodiments the sample preparation module 120 includes a reservoir to retain, for example, an extraction agent (e.g. organic solvent) and/or other reagents. In some embodiments, the organic solvent and/or other reagents (e.g. hydrolyzing and SPM enzymes) are not contained in a reservoir but instead are crystallized or lyophilized or in some other way stored in or on the substrate 102 (e.g. membrane) and then reconstituted or activated when exposed to the sample. In other embodiments, sample preparation module 120 may include two or more submodules, each including zero, one or more reservoirs for retaining organic solvent and/or a hydrolyzing agent or other reagents.

In the embodiment shown in FIG. 1, sample preparation module 120 includes a reservoir 122 and a microchannel 124 connecting the reservoir 122 to the detection module 130. Again, reservoir 122 and microchannel 124 can be formed into the substrate 102 in any manner discussed previously with respect to microchannel 104. Within the sample preparation module 120 the fluid sample may undergo one or more processes to form a prepared fluid sample. The one or more processes may include but are not limited to an extraction process, a hydrolysis process and a conversion process to provide the prepared fluid sample to the detection module 130. Each of an extraction process, a hydrolysis process and a conversion process for forming the prepared fluid sample is described in greater detail below. Each of the extraction process, the hydrolysis process and the conversion process may take place in a single reservoir of the sample preparation module, in a reservoir with one or more other processes, or in an independent reservoir. In some embodiments, the sample preparation module 120 may include submodules for one or more of the extraction process, the hydrolysis process and the conversion process to occur.

In some embodiments, the prepared fluid sample includes Resolvin D1 (RvD1). Accordingly, the reservoir(s) of the sample preparation module 10, such as but not limited to reservoir 122, may be configured to retain one or more fluids to be combined with the fluid sample received from the microchannel 104 to conduct one or more of processes for preparing the prepared fluid sample. As noted above, the substrate 102 may also be configured to retain one or more fluids to be combined with the fluid sample to conduct one or more of processes for preparing the prepared fluid sample.

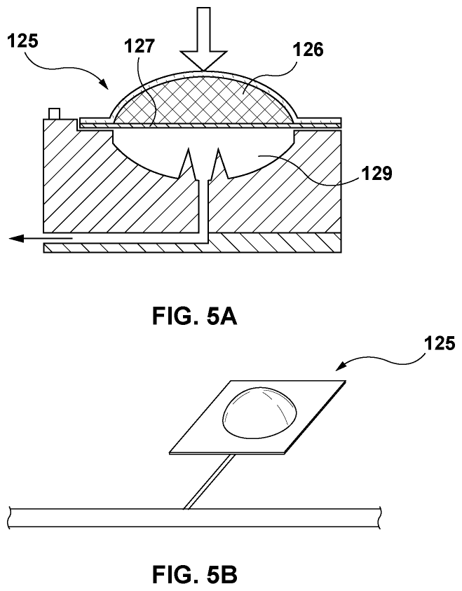

In one embodiment, storage and retaining of fluids in reservoir 122 of sample preparation module 120 (and in detection module 130, described below) to be combined with the fluid sample to form the prepared fluid sample can be by usage of a blister 125. An example of a blister 125 is shown in FIGS. 5A and 5B. Generally, blister 125 is a separate chamber containing a defined volume of a fluid 126 that is fluidly connected to the rest of the microfluidic device 100 (e.g. fluidly connected to a reaction chamber 129 of reservoir 122), but the connection is obstructed by a barrier 127. Connection of the fluid 126 to the reaction chamber 129 can be restored by disruption of the barrier 127 between the fluid 126 and reaction chamber 129. In some embodiments, the barrier 127 may be thin aluminum foil.

In another example, another way to retain fluid in the reservoir 122 is to reduce the flow velocity of the sample fluid. This may be achieved either by varying the diameter of the microchannels or by application of membranes with different properties (e.g. porosity).

In another example, it may be appropriate to store some chemicals directly in the reservoir 122 as a lyophilizate. Lyophilizates may also be stored on a surface (e.g. a membrane) within reservoir 122. Lyophilizates could be solubilized when brought in contact with the fluid sample (or another fluid).

Returning to the embodiment of microfluidic device 100 shown in FIG. 1, as shown therein, detection module 130 is fluidly coupled to the sample preparation module 120 and receives at least a portion of the prepared fluid sample from the sample preparation module 120. In the detection module 130, the prepared fluid sample undergoes a bioassay that quantifies a component of the prepared fluid sample (e.g. the RvD1). For instance, in some embodiments, the bioassay may include using an RvD1 antibody in a competitive ELISA (or sandwich ELISA) to quantify RvD1 in the fluid sample. In other embodiments, the bioassay may include using an RvD1 aptamer to quantify RvD1 in the fluid sample. In other embodiments, the bioassay may include using RvD1 antibody or aptamer functionalized onto a cantilever biosensor to quantify RvD1 in the fluid sample.

In some embodiments, the bioassay that is performed in the detection module 130 can result in a color change of a component within the detection module 130. The color change may be visualized by a user of the microfluidic device 100 and used to quantify fatty acids in the fluid sample. For instance, in some embodiments, streptavin may be conjugated to a reporter molecule (e.g. horseradish peroxidase) and bound to biotin to induce a color change that can be visualized by a user of the microfluidic device 100. In some embodiments, a label may be used such as gold nanobeads, cellulose nanobeads, or latex nanobeads.

In some embodiments, other mechanisms can be used to quantify fatty acids and/or fatty acid metabolites in the fluid sample. For instance, electrical readouts based on cantilever biosensors covered in RvD1 antibodies may be used. Electrical readouts may be based on a piezoresistive effect of the cantilevered biosensor, such as a change in the electrical resistivity of a semiconductor or metal forming the cantilevered biosensor when mechanical strain is applied (e.g. the free fatty acid is bound thereto).

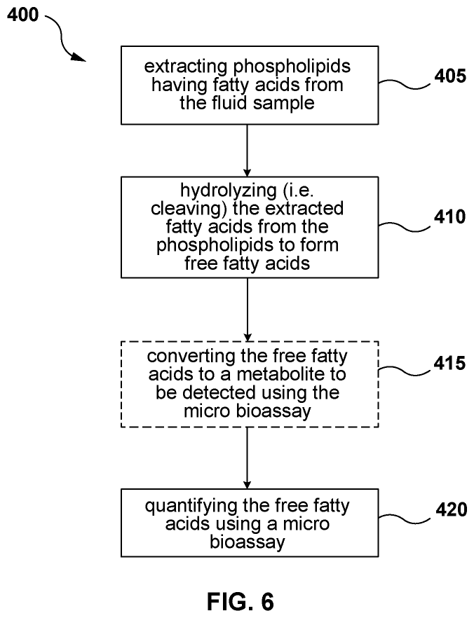

Referring now to FIG. 6, illustrated therein is a method 400 of quantifying fatty acids and/or fatty acid metabolites in a fluid sample. Method 400 includes, at a step 405, extracting fatty acid esters (e.g. triglycerides, phospholipids and/or cholesteryl esters)) having fatty acids from the fluid sample, at a step 410, hydrolyzing (i.e. cleaving) the fatty acids from the extracted fatty acid esters to form free fatty acids; and, at a step 420, quantifying the free fatty acids using a micro bioassay. In some embodiments, the method 400 also includes, at a step 415, converting the free fatty acids to a fatty acid metabolite to be detected using a bioassay. Each of these steps is described in detail below.

It should be understood that, in some embodiments, methods of quantifying fatty acids in a fluid sample described herein may include hydrolyzing fatty acids from fatty acid esters and converting the free fatty acids to a fatty acid metabolite (such as RvD1) without extracting the fatty acid esters from the fluid sample.

It should also be understood that, in some embodiments, the methods described herein may also be used to quantify fatty acid metabolite(s) levels in a fluid, the fatty acid metabolite(s) including but not limited to fatty acid metabolites that are present in the following groups of metabolites: SPMs, prostaglandins, endocannabinoids, eicosanoids, leukotrienes and lipoxins.

It should also be understood that, in some embodiments, the methods described herein may also be used to quantify DHA metabolites, including but not limited to DHA-derived SPMs, DHA epoxides, electrophilic oxo-derivatives (EFOX) of DHA, neuroprostanes, ethanolamines, acylglycerols, docosahexaenoyl amides of amino acids or neurotransmitters, and branched DHA esters of hydroxy fatty acids.

It should also be understood that, in some embodiments, the methods described herein may also be used to quantify fatty acid metabolites including omega-3 mediators that may be used as biomarkers for omega-3 fatty acid levels or for detection themselves independent of omega-3 fatty acid levels (e.g. measuring endocannabinoid levels for sake of measuring endocannabinoid levels and making a health claim based upon endocannabinoid levels, not omega-3 levels), either by direct detection, or by conversion, enzymatically or chemically. These fatty acid metabolites including omega-3 mediators may include but are not limited to: 2-Arachidonoylglycerol (2-AG), 2-Arachidonoylglycerol-lysophosphatidic acid (2-AG-LPA), 2-Arachidonoyl-lysophosphatidylinositol (2-AG-LP I), 2-Docosahexaenoylglycerol (2-DHG), 2-Epoxy-eicosatrienoic acid glycerol (2-EET-EG), 2-Eicosapentaenoylglycerol (2-EPG), Adrenic acid (AdA), N-arachidonoylethanolamide (anandamide) (AEA), N-docosahexaenoylethanolamine (synaptamide) (DHEA), Dihydroxy-docosahexaenoic acid (DiHDoHE), Dihydroxy-docosapentaenoic acid (DiHDPE), Dihydroxy-eicosapentaenoic acid (DiHEPE), Dihydroxy-eicosatetraenoic acid (DiHETE), Dihydroxy-eicosatrienoic acid (DiHETrE), Epoxy-docosapentaenoic acid (EDP) Epoxy-eicosatrienoic acid (EET), Epoxy-eicosatrienoic acid ethanolamide (EET-EA), Epoxy-eicosatetraenoic acids (EETeTr), Electrophilic fatty acid oxo-derivative (EFOX), Eicosapentaenoic acid (EPA), Epoxy-docosapentaenoic acid (EpDPE), N-eicosapentaenoylethanolamine (EPEA), Epoxy-eicosapentaenoic acid (EpETE (EEQ)), Epoxy-eicosatrienoic acid (EpETrE), Glycerophosphoarachidonoylethanolamide (GP-NAPE), Hydroxy-docosahexaenoic acid (HDoHE), Hydroxy-epoxy-docosapentaenoylethanolamide (HEDPEA), Hydroxy-epoxy-eicosatrienoic acid ethanolamide (HEET-EA), Hydroxy-eicosapentaenoic acid (HEPE), Hydroxy-eicosatetraenoic acid (HETE), Hydroxy-eicosatetraenoic acid ethanolamide (HETE-EA), Hydroxy-heptadecatrienoic acid (HHTrE), 4-Hydroxy hexenal (HHE), Hydroperoxy-docosahexaenoic acid (HpDoHE), Hydroperoxy-eicosapentaenoic acid (HpEPE), Hydroperoxy-eicosatetraenoic acid (HpETE), Hepoxilin (Hx), Maresin (MaR), (Neuro)protection D1 ((N)PD1), N-acyl phosphatidylethanolamine-selective phospholipase D (NAPE-PLD), N-arachidonoyl phosphatidylethanolamine (NArPE), Oxo-eicosatetraenoic acid (oxo-EET), Phospho-anandamide (PAEA), Protectin (PD), Phosphodiesterase (PDE), Phosphatidylethanolamine (PE), Prostaglandin D metabolite (PGD), Prostaglandin E metabolite (PGE), Prostaglandin F metabolite (PGF), Prostaglandin E, D or F or prostacyclin synthase (PGS), Resolvin D series (RvD), Resolvin E series (RvE), and Stearidonic acid (SDA).

### Extraction

To quantify fatty acids (e.g. omega-3 or omega-6 fatty acids) and/or fatty acid metabolites in the fluid sample, in some embodiments, the fluid sample may be exposed to (i.e. combined with), at a step 405, an extraction agent (e.g. such as but not limited to an organic solvent, iPLA2, iPLA2-β, or the like) to extract (i.e. remove) fatty acid esters having the fatty acids from the fluid sample. In some embodiments, the fluid sample is a blood sample and combining the blood sample with an extraction agent extracts the fatty acid esters from the plasma of the blood sample.

In some embodiments, the extraction solvent and the fluid sample may be mixed for the extraction to occur. Mixing the extraction solvent and the fluid sample may be by passive mixing (e.g. in a microchannel) or by active mixing.

In some embodiments, the efficiency and reliability of the extraction process and the time required to complete the extraction process may be controlled by heating or cooling the fluids during the extraction process. Accordingly, the sample preparation module 120 may be configured to be heated and/or cooled during the extraction step 405.

In some embodiments, the step 405 occurs after a volume of the fluid sample has been normalized in the microchannel 104.

During step 405, the fluid sample is received by the sample preparation module 120 of the microfluidic device 100. In the sample preparation module 10, the fluid sample (e.g. plasma) is combined with an extraction solution retained in the sample preparation module 120 (e.g. reservoir 122) to extract fatty acid esters containing the one or more fatty acids present in the fluid sample.

In some embodiments, the extraction solution includes an organic solvent to extract the fatty acid esters containing the fatty acids. Various solvents may be used, including but not limited to organic solvents such as organic solvents appropriate/used in the Folch extraction method (e.g. a mixture of chloroform and methanol in a ratio of about two parts chloroform to about one part methanol (e.g. by weight) or a similar ratio of a mixture of dichloroform and methanol) and organic solvents appropriate/used in the Bligh Dyer extraction method (e.g. a mixture of about one part chloroform to about two parts methanol (e.g. by weight)).

It should be noted that both of the Folch extraction method and Bligh Dyer extraction method mentioned above extract lipids from a solution based on chloroform and methanol (MeOH) by disrupting hydrogen bonds or electrostatic forces between proteins and lipids. Accordingly, any organic solvent capable of extracting lipids in this manner may also be appropriate for extracting the fatty acids (e.g. omega 3 or omega-6 fatty acids) from the fluid (e.g. plasma) such as but not limited to: an acidified Bligh and Dyer extraction method; the Dole method (e.g. 1 mL plasma to 5 mL Dole reagent (e.g. 40 mL isopropanol, 10 mL heptane and 1 mL 1M H₂SO₄), 3 mL heptane, 2 mL water); a mixture of about 2:1 chloroform:methanol; a mixture of about 20:10:1 hexane:dichloromethane:2-propanol; a mixture of BF₃-MeOH; butanol; eythyl acetate; sodium methoxide; Radin's method (e.g. about 3:2 hexanes:isopropanol); methanol-tert-butylmethyl ether (MTBE); a MeOH single step method (e.g. 2 μL of plasma or serum added into 1 mL of MeOH); n-hexane; Sigma Aldrich fatty acid extraction kit applied on chip; and a one-step method (e.g. Lapage and Roy A 1-hour direct transesterification procedure carried out in methanol-benzene 4:1 with acetyl chloride circumvented all these steps and was applicable for analysis of both simple (triglycerides) and complex lipids (cholesteryl esters, phospholipids; and sphingomyelin)).

In some embodiments, the extraction of the fatty acid esters containing the fatty acids is achieved without the use of an organic solvent using methods such as, but not limited to, osmotic pressure, isotonic extraction, enzyme-assisted extraction, and/or ionic liquids, or any combination of one or more.

In some embodiments, after extraction step 405, the fluid sample may be enriched to result in a sample containing more fatty acid esters and less biological debris from the plasma/blood/fluid sample. Enrichment of the fluid sample after extraction may be achieved using a phospholipid cartridge embedded in the microfluidic device 100. For example, a HybridSPE®-Phospholipid Technology may be used. In this example, the following method, according to one embodiment, may be used to enrich the fluid sample: i) the cartridge may be conditioned with a formic acid solution (e.g. 1 wt %) in acetonitrile; ii) the fluid sample can be mixed with a 1 wt % formic acid solution in acetonitrile; iii) 100 uL of plasma can be added to an enrichment filter combined with 300 uL of precipitation solvent; iv) the solution is mixed or agitated; v) the mixed or agitated solution is drawn through a filter to separate fatty acid esters from the solution; vi) fatty acid esters are washed out of the filter with a complexant and/or a base such as but not limited to a 5% ammonium hydroxide solution; and adding methanol to the fatty acid esters.

### Hydrolysis

Once the fatty acid esters containing the fatty acids have been extracted from the fluid sample, at step 410, the extracted fatty acid esters are hydrolyzed to cleave the fatty acids from the fatty acid esters and generate free fatty acids. In some embodiments, both of step 410 and step 405 occur in a reaction chamber of the sample preparation module 120. In some embodiments, step 410 and step 405 occur in separate reaction chambers of the sample preparation module 120.

In some embodiments, the extracted fatty acid esters are hydrolyzed by a hydrolysis agent to cleave the fatty acids from the fatty acid esters. In some embodiments, the hydrolysis agent can selectively cleave fatty acids from the fatty acid esters. For example, the hydrolysis agent may selectively cleave one or more omega-3 fatty acids from the extracted fatty acid esters. For example, the hydrolysis agent may selectively cleave one or more of AA, EPA and DHA from the extracted fatty acid esters.

In some embodiments, the hydrolysis agent can be an enzymatic hydrolysis agent to enzymatically cleave one or more fatty acids from the extracted fatty acid esters. The enzymatic hydrolysis agent may also be retained in the sample preparation module 120 of the microfluidic device 100. For instance, the enzymatic hydrolysis agent may be retained in reservoir 122 of microfluidic device 100.

It should be understood that omega-3 fatty acids may be selectively cleaved from fatty acid esters based on the position of the fatty acids on the phospholipid and the tendency of different phospholipase enzymes to attack different phospholipid bonds. For instance, omega-3 fatty acids are typically located at the sn-2 position of the fatty acid esters. Phospholipase A's are classified based on the acyl-ester bond they attack. Since omega-3s tend to be located on the sn-2 position of the phospholipid, PLA2 enzymes may be used to generate free omega-3 fatty acids. Phospholipase B (PLB), also known as lysophospholipase, enzymes may also be used as they can attack both phospholipid bonds.

Lipases are naturally occurring enzymes (as in humans) and they also hydrolyze fatty acid esters. In some embodiments, lipases may be used in combination or separately from phospholipases to generate free fatty acids.

In some embodiments, free fatty acids may be cleaved from the extracted fatty acid esters by enzymatically hydrolyzing the extracted fatty acid esters within the sample preparation module 120. This may occur by combining the enzymatic hydrolysis agent with the extracted fatty acid esters and, optionally, mixing.

In these embodiments, enzymatic hydrolysis of the extracted fatty acids may occur at high temperatures. For instance, the chemical hydrolysis may occur in a range of about 10° C. to about 100° C., or in a range of about 20° C. to about 50° C., or in a range of about 30° C. to about 40° C., or at a temperature of about 37° C.

In some embodiments, enzymes that may be appropriate for use in enzymatic hydrolysis agents include but are not limited to: lipase, phospholipase, phospholipase A, phospholipase A2, cPLA2, iPLA2, sPLA2, Phospholipase A2 from honey bee venom, phospholipase D, Burkholderia cepacia (BC) lipase Chromobacterium viscosum lipase, Lipase A Candida antarctica, Rhizopus oryzae (RO) lipase, Pancreatin Lipase, lipoxygenases (e.g. LOX) such as 5-LOX-5, 12-LOX, 15-LOX (ALOX15 or ALOX15B), human ALOX15-2 and combination of pancreatin lipase and PLA2 (1:1) as per the Diteba experiment. Other enzymes may include but are not limited to CYp450 Cytochrome P450 enzymes, specifically the Cyp1 family of cytochrome p450 enzymes. This includes but is not limited to CYP1A2, CYP2C8, CYP2C9, CYP2D6, CYP2E1, and/or CYP3A4. Other enzymes may include but are not limited to hydrolases, including soluble epoxide hydrolase (sEH), glutathione S-transferase (GST) and several members of the cytochrome P450 superfamily (epoxydases, u-hydrolases), 15-hydroxyprostaglandin dehydrogenase, 15PGDH, fatty acid amide hydrolase (FAAH),

In some embodiments, enzymes that may be appropriate for use in enzymatic hydrolysis agents include but are not limited to: cyclooxygenases, (e.g. COX) such as COX-2, COX-1,

In some embodiments, a combination of pancreatin lipase and phospholipase A2 may be appropriate for including in the hydrolyzing agent in the methods and devices described herein. For instance, an enzyme mixture prepared by the following method may be appropriate as a hydrolyzing agent in the methods and devices described herein: preparing a pancreatin lipase mixture by: i) adding 20 mg of pancreatin lipase into a vial; ii) adding 500 uL of buffer to the vial; and iii) mixing the contents of the vial; and preparing a phospholipase A2 mixture by: i) weighing ˜5 mg of phosphatelipase A2 into vial; ii) adding 500 uL of buffer to the vial; and iii) mixing the contents of the vial. The resulting enzyme mixture can then be a 1:1 phospholipase A2 mixture: pancreatin lipase mixture (e.g. mix 200 uL pancreatin lipase solution and 200 uL of phosphatelipase A2).

Once the enzyme mixture described above has been prepared, the hydrolyzing agent can be prepared as a 100 uL:100 uL:50 uL (i.e. 2:2:1) mixture of plasma: buffer solution: enzyme mixture. In some embodiments, the buffer solution of the hydrolyzing agent can be a digestion buffer: 0.1M Tris-HCl, having a pH of 9.0.

In some embodiments, the hydrolysis agent can be a chemical hydrolysis agent to chemically cleave one or more fatty acids from the extracted fatty acid esters. The chemical hydrolysis agent may be retained in the sample preparation module 120 of the microfluidic device 100. For instance, the chemical hydrolysis agent may be retained in reservoir 122 of microfluidic device 100.

In these embodiments, chemical hydrolysis of the extracted fatty acids may occur at high temperatures. For instance, the chemical hydrolysis may occur in a range of about 10° C. to about 100° C., or in a range of about 20° C. to about 50° C., or in a range of about 30° C. to about 40° C., or at a temperature of about 36° C.

In some embodiments, the microfluidic device 100 may include one or more microheaters to increase the temperature of the device for chemical hydrolysis.

In other embodiments, external heaters may be used to provide heat to the microfluidic device 100 or heating resistors or the use of electromagnetic radiation may be integrated into the microfluidic device 100 to increase the temperature of the microfluidic device 100. For instance, Peltier elements may be integrated into the microfluidic device 100. In another example, a Joule heating temperature control method could be used such as but not limited to Joule heating of ionic liquids with an AC current.

In another embodiment, microwaves may be used to heat the microfluidic device 100.

In another embodiment, microfluidic device 100 may include one or more reservoirs (e.g. positioned adjacent to and/or below the sample preparation module) to house one or more exothermic chemical reactions to provide heat to the microfluidic device 100. For example, the dissolution of 97 wt % H₂SO₄ (Reagent 1) in water (Reagent 2) could be used to provide heat to the microfluidic device 100.

Similarly, in some embodiments, chemical hydrolysis of the extracted fatty acids may include hydrolysis by one or more of potassium hydroxide (KOH), sodium hydroxide (NaOH), deacylation with lithium hydroxide in chloroform-methanol (e.g. having a ratio in a range of about 2:8), mild alkaline, and/or hydrochloric acid (HCl).

In some embodiments, the chemical hydrolyzing agent can be a mixture having a ratio of 4:6 of chloroform and methanol, respectively, with about 0.1 M (e.g. 0.01-1 M) base. The base may be one of, but not limited to being one of, the following bases: KOH, phosphate ester, NaOH, LiOH, NaOCH₃, LiOCH₃, and Et₄NOH.

In some embodiments, the chemical hydrolyzing agent can be a mixture having a ratio of 2:3 methanol and water, respectively, in 0.1M NaOH

In some embodiments, the chemical hydrolyzing agent can be a mixture having a ratio of 2:3 chloroform and ethanol, respectively, in 0.1M NaOH

Enzymatic hydrolysis agents generally require milder conditions than chemical hydrolysis agents that may be used to cleave fatty acids from the extracted fatty acid esters. Further, enzymatic hydrolysis agents may more accurately selectively cleave polyunsaturated fatty acids like omega-3 fatty acids from the extracted fatty acid esters.

### Conversion

In some embodiments, following extraction of the fatty acid esters at step 405 and the hydrolysis of the fatty acids at step 410, it may be necessary to convert the free fatty acids to a fatty acid metabolite that can be quantified using a bioassay at a step 415.

For instance, in some embodiments when the free fatty acid formed during step 410 is DHA, the DHA can be converted to Resolvin D1 (RvD1), 17(R)-hydroxyl or 17(S)-hydroxyl, at step 415.

In other embodiments, when the free fatty acid formed during step 410 is DHA, the free DHA may be converted at step 415 to another fatty acid metabolite that can be quantified using a bioassay, such as but not limited to: 17-hydroxy DHA, 14-hydroxy DHA Resolvin D2 (RvD2), Resolvin D3, Resolvin D4, Resolvin D5, Resolvin D6, Maresin 1 (Mar 1), Maresin 2, isofurans, isoprostanes and PDX, Neuroprotectin D1 (NPD1), PD1n-3 (10,17-dihydroxy-7,11,13,15,19-DPA), PD2n-3 (16,17-dihydroxy-7,10,12,14,19-DPA), or the aspirin-triggered form of these SPMs (e.g. At-RvD1 or the like). It should be understood that, if SPMs are created in the presence of ASA and Cox-2 they form aspirin-triggered SPMs. In some embodiments, aspirin-triggered SPMs are more stable then non-aspirin-triggered SPMs. All resolvins (e.g. D and E series resolvins) may be present in their aspirin triggered forms.

In yet other embodiments, the free fatty acid formed during step 410 may not need to be converted to a fatty acid metabolite to be detected by a bioassay. For instance, when the free fatty acid is one or more of AA, DHA, DPA or EPA, or one of their metabolites, it may not be necessary to convert the AA, DHA, DPA or EPA to be detected by a bioassay. In some embodiments, the target for EPA may include but is not limited to Resolvin E1, Resolvin E2, Resolvin E3, or 18-HEPE, or the aspirin triggered form of these SPMs (eg. At-RvE1).

In some embodiments the free fatty acid formed during step 410 may be conjugated to another molecule to facilitate the detection and quantification of the fatty acid.

In some embodiments the fatty acid (e.g. DHA) may be conjugated to coenzyme A (e.g. CoA), rhodopsin, Arecoline, or conjugated DHA (e.g. CDHA—conjugated fatty acids are positional and geometrical isomers with conjugated double bonds) or the like. In some embodiments, fatty acid conjugation may be by enzyme Acyl-CoA synthetase 6 (Acsl6).

In some embodiments, a solution containing free DHA can be exposed to an enzymatic solution to convert free DHA to RvD1. In some embodiments, the solution containing free DHA moves to a subsequent reservoir on the microfluidic device 100 to be exposed to an enzymatic solution to convert free DHA to RvD1. In other embodiments, a solution containing free DHA may be combined with an enzymatic solution within the reservoir 122 of the microfluidic device 100 to convert the free DHA to RvD1.

It should be noted that conversion at step 415 may occur in the same reaction chamber as the extraction and the hydrolysis processes described above. Alternatively, the hydrolysis and conversion processes may occur in the same reaction chamber and extraction may require a separate chamber. Alternatively again, the extraction and hydrolysis processes may occur in the same reaction chamber and the conversion process may require a separate chamber. Alternatively again, all three of the extraction, hydrolysis and conversion processes may occur in separate reaction chambers.

Further, as noted above with respect to the sample preparation module 10, chemicals used in any one or more of the extraction, hydrolysis and/or conversion processes may be housed and stored in the reaction chamber or may be stored in a reservoir fluidly coupled to the reaction chamber and combined with the sample in the reaction chamber.

In some embodiments, DHA present in the fluid sample can be converted to RvD1 using a “one-pot” reaction. Herein, the term “one-pot” reaction refers to a reaction in which both enzymatic conversions occur in the same reaction chamber (e.g. both enzymes are present at the same time. It should be noted that the DHA present in the fluid sample undergoes several reactions in the same chamber as it becomes RvD1

For instance, the DHA present in the fluid sample can be converted to RvD1 using: potato 5-LOX or soybean LOX (type IV; from Sigma). In some embodiments, when soybean LOX is used, DHA (e.g. 2 mg) can be incubated with soybean LOX (100 kilounits, 701 kilounits/mg of protein, 3.6 mg of protein/mL) in borate buffer (5 mL, pH 9.3) at 4° C. to convert the DHA to RvD1.

### Bioassay

In some embodiments where conversion of free fatty acids in solution to a fatty acid metabolite to be quantified is not necessary, the free fatty acids may be quantified directly by performing a bioassay.

In some embodiments, SPMs or fatty acid metabolites may be quantified directly without conversion of omega-3 fatty acids. This method may not require a sample reservoir and reaction chamber. This embodiment may only require sample normalization and then exposing the normalized sample, whole blood or plasma, to the SPM bioassay. This mechanism may be used to quantify SPMs directly or to quantify omega-3s as a biomarker.

In some embodiments where conversion of free fatty acids in solution to a fatty acid metabolite to be quantified is necessary, a fatty acid metabolite of the free fatty acids may quantified by performing a bioassay.

For instance, in some embodiments, to detect free EPA and free AA in the solution, an antibody unique to EPA or AA could be used to detect each respective fatty acid in solution. In other embodiments, an antibody unique to a metabolite of EPA or AA could be used to detect each respective fatty acid in solution. Such metabolites may include but are not limited to: Resolvin E series (i.e. RvEs are di- or tri-hydroxyl metabolites of EPA; four RvEs have been described to date: RvE1 (5S,12R,18R-trihydroxy-EPA), 185-Rv1 (5S,12R,18S-trihydroxy-EPA), RvE2 (5S,18R-dihydroxy-EPA) and RvE3 (17R,18R/S-dihydroxy-EPA), 18-HEPE (; EFOX, leukotrienes, thromboxanes, prostaglandins and prostacyclin.

In other embodiments, other potential biorecognition methods other than antibodies may be used to detect each respective fatty acid in solution. For example, these other potential biorecognition methods may include the use of aptamers to detect and/or quantify each respective fatty acid in solution.

In other embodiments, after the free fatty acid (e.g. DHA) is conjugated (itself or to another molecule) at step 415, a solution containing the conjugated DHA may be exposed to a micro bioassay at 420 in order to quantify the amount of conjugated DHA and thereby the amount of DHA.

In some embodiments, SPMs or fatty acid metabolites may be conjugated to detect and/or quantify either SPMs or fatty acids (e.g. omega-3 fatty acids).

In other embodiments, after the free fatty acid (e.g. DHA) is converted to a fatty acid metabolite to be quantified (e.g. RvD1) at step 415, a solution containing the fatty acid metabolite maybe exposed to a micro bioassay to quantify the fatty acid metabolite at 420. In some embodiments, the micro bioassay contains a commercially available antibody against the fatty acid metabolite to be quantified. For instance, when the fatty acid metabolite is RvD1, the micro bioassay includes an antibody that captures free RvD1 and a colorimetric response is generated in proportion to the amount of RvD1 present in the sample.

In some embodiments, a colorimetric response is generated during the micro bioassay in proportion to the amount of fatty acid (or fatty acid metabolite) present in the sample.

For instance, in examples where RvD1 is being detected, an ELISA test can may be performed to generate a colorimetric response. The type of ELISA test performed may depend on the microbioassay.

For example, a competitive ELISA may be performed to generate a colorimetric response where RvD1 derived from the fluid sample will displace RvD1 preloaded in the bioassay and tagged with a fluorescent or colorimetric. In this example, a level of RvD1 in the fluid sample is inversely proportional to the signal (i.e. the more RvD1 from the user's blood sample, the lower the signal will be (less bound tagged RvD1)).

In another example, a sandwich ELISA may be performed to generate a colorimetric response where RvD1 from the fluid sample will bind to an RvD1 antibody. The bound RvD1 will bind to a second antibody that is tagged (e.g. with biotin). In this example, a visible colorimetric inducing reagent such as but not limited to horseradish peroxidase (HRP)—Streptavidin reacts with the biotin in proportion to the amount of RvD1 bound by the bioassay.

In another example, a plasmonic ELISA may be performed to generate a colorimetric response where horseradish peroxidase (HRP) and hydrogen peroxide (H₂O₂) and tyramine (TYR)-induced gold nanoparticle (AuNP) aggregation can be used as a signal output. AuNP aggregation may be triggered through phenol polymerization of TYR, which may be induced by hydroxyl radicals from HRP-catalyzed H₂O₂. DHA-labeled catalase (CAT) can be used as a competing antigen to consume H₂O₂.

In another example, a multi-colour colorimetric immunoassay may be performed. Here, a concentration-dependent multicolor conversion strategy based on gold nanoparticle (AuNP)-mediated copper deposition for signal amplification and Prussian blue for color generation may be used.

In another example, the secondary antibody may be labeled with a gold nanoparticle, latex nanobeads or cellulose nanobeads which produce a colorimetric response visible to the naked eye and/or quantifiable by a cell phone spectrometer.

A user may interpret the color generated during the colorimetric response to quantify the amount of fatty acids in the fluid sample. In some embodiments, the color change may reflect if the fluid sample has high/medium/low DHA levels (semi-quantitative). In some embodiments, the colour change may be a fully quantitative readout based on a color scale or a digital readout using a biosensor technology.

In another example the color change may reflect a cut-off point (qualitative). This cut-off point may represent sufficiency/deficiency or a specific concentration (e.g. of SPMs or omega-3 fatty acids or fatty acid metabolites, such as but not limited to a concentration of about 40 μg/mL).

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. A method of quantifying fatty acids present in a fluid sample on a microfluidic device, the method comprising:
extracting fatty acid esters containing the fatty acids from the fluid sample;
combining the extracted fatty acid esters with a hydrolyzing agent on the microfluidic device to cleave the fatty acids from the extracted fatty acid esters and form free fatty acids; and
quantifying the free fatty acids by performing a bioassay specific to the free fatty acids on the microfluidic device.

2. A method of quantifying a specialized pro-resolving mediator present in a fluid sample on a microfluidic device, the method comprising:
extracting fatty acid esters containing fatty acids from the fluid sample;
combining the extracted fatty acid esters with a hydrolyzing agent on the microfluidic device to cleave fatty acids from the extracted fatty acid esters and form free fatty acids;
converting the free fatty acids to the specialized pro-resolving mediator to be detected in a bioassay; and
quantifying the specialized pro-resolving mediator by performing a bioassay specific to the specialized pro-resolving mediator on the microfluidic device.

3. A method of quantifying a fatty acid metabolite present in a fluid sample on a microfluidic device, the method comprising:
extracting fatty acid esters from the fluid sample;
combining the extracted fatty acid esters with a hydrolyzing agent on the microfluidic device to cleave fatty acids from the extracted fatty acid esters;
converting the free fatty acids to the fatty acid metabolite to be detected in a bioassay; and
quantifying the fatty acid metabolite by performing a bioassay specific to the fatty acid metabolite on the microfluidic device.

4. The method of any one of claims 1 to 3, wherein extracting the fatty acid esters from the fluid sample includes combining the fluid sample with an extraction agent stored on the microfluidic device.

5. The method of claim 4, wherein the extraction agent is an organic solvent.

6. The method of claim 1, further comprising, after combining the extracted fatty acid esters with the hydrolyzing agent on the microfluidic device to form the free fatty acids:
converting the free fatty acids to a fatty acid metabolite to be detected in the bioassay; and
quantifying the free fatty acids by detecting the fatty acid metabolite with an antibody unique to the fatty acid metabolite.

7. The method of claim 6, wherein the free fatty acids include DHA and the DHA is converted to RvD1.

8. The method of claim 7, wherein the DHA is converted to RvD1 by combining the DHA with an enzyme.

9. The method of claim 8, wherein the enzyme is selected from a group consisting of: 5-lipoxgenase (5-LOX), soybean lipoxgenase, 12-lipoxgenase (12-LOX), 15-lipoxgenase (15-LOX), human ALOX15-2, cyclooxygenase-1 (COX-1), cyclooxygenase-2 (COX-2), Cy P450, a combination of 15-LOX and 5-LOX, a combination of 12-LOX and 15-LOX and a combination of cyclooxygenase-2 (COX-2) and acetylsalicylic acid (ASA).

10. The method of claim 1, further comprising, after combining the extracted fatty acid esters with the hydrolyzing agent on the microfluidic device to form the free fatty acids:
conjugating the free fatty acids to be detected in the bioassay; and
quantifying the free fatty acids by detecting the conjugated fatty acids with an antibody unique to the conjugated fatty acid.

11. The method of any one of claims 1 to 10, wherein the hydrolyzing agent is a chemical hydrolyzing agent.

12. The method of claim 11, wherein the chemical hydrolyzing agent comprises one or more of the following: potassium hydroxide (KOH), sodium hydroxide (NaOH), deacylation with lithium hydroxide in chloroform-methanol, and/or hydrochloric acid (HCl)

13. The method of any one of claims 1 to 10, wherein the hydrolyzing agent is an enzymatic hydrolyzing agent.

14. The method of claim 13, wherein the enzymatic hydrolyzing agent is selected from a group consisting of: lipases, phospholipases, phospholipases A (PLA), phospholipase A2 (PLA2s), Ca2+-independent phospholipase A2 (iPLA2 β), cytosolic phospholipases A2 (cPLA2s), lipoprotein-associated phospholipases A2 (Ip-PLAs2), secreted phospholipases (sPLA2s), phospholipases D, Burkholderia cepacia (BC) lipase Chromobacterium viscosum lipase, Lipase A Candida antarctica, Rhizopus oryzae (RO) lipase, pancreatin lipase, and a combination of two or more of any of the above.

15. The method of any one of claims 1 to 14 further comprising, prior to combining the fluid sample with an organic solvent, normalizing a volume of the fluid sample.

16. The method claim 4, further comprising, after combining the fluid sample with an extraction agent, normalizing a sample volume containing extracted fatty acid esters.

17. The method of any one of claims 1 to 16 further comprising, filtering the fluid sample into red blood cells and plasma prior to normalizing a volume of the fluid sample.

18. The method of any one of claims 1 to 16 further comprising, filtering the fluid sample into red blood cells and plasma after normalizing a volume of the fluid sample.

19. The method of any one of claims 1 to 18, wherein the bioassay generates a colorimetric response to quantify the free fatty acids, the specialized pro-resolving mediator or the metabolite.

20. The method of any one of claims 1 to 18, wherein the bioassay generates an electrochemical response to quantify the free fatty acids, the specialized pro-resolving mediator or the metabolite.

21. The method of any one of claim 1 to claim 18, wherein the bioassay generates a response detectable by a cellular phone spectrophotometer.

22. A microfluidic device for quantifying fatty acids in a fluid sample, the microfluidic device comprising:
a sample preparation module configured to receive the fluid sample, the sample preparation module being configured to:
extract fatty acid esters containing the fatty acids from the fluid sample; and
retain a hydrolyzing agent to cleave the one or more fatty acids from the extracted fatty acid esters and form free fatty acids; and
a detecting module fluidly coupled to the sample preparation module and configured to receive the free fatty acids from the sample preparation module;
wherein one of the sample preparation module and the detecting module retains an assay agent to be bound to the free fatty acids to quantify the free fatty acids in the fluid sample.

23. A microfluidic device for quantifying a specialized pro-resolving mediator in a fluid sample, the microfluidic device comprising:
a sample preparation module configured to receive the fluid sample, the sample preparation module having a reservoir configured to:
extract fatty acid esters containing one or more fatty acids from the fluid sample; and
retain a hydrolyzing agent to cleave the one or more fatty acids from the extracted fatty acid esters and form free fatty acids; and
a detecting module fluidly coupled to the sample preparation module and configured to receive the free fatty acids from the sample preparation module;
wherein one of the sample preparation module and the detecting module retains an assay agent to be bound to the free fatty acids to quantify the specialized pro-resolving mediator in the fluid sample.

24. A microfluidic device for quantifying a fatty acid metabolite in a fluid sample, the microfluidic device comprising:
a sample preparation module configured to receive the fluid sample, the sample preparation module having a reservoir configured to:
extract fatty acid esters containing one or more fatty acids from the fluid sample; and
retain a hydrolyzing agent to cleave the one or more fatty acids from the extracted fatty acid esters and form free fatty acids; and
a detecting module fluidly coupled to the sample preparation module and configured to receive the free fatty acids from the sample preparation module;
wherein one of the sample preparation module and the detecting module retains an assay agent to be bound to the fatty acid metabolite to quantify the fatty acid metabolite in the fluid sample.

25. The microfluidic device of any one of claims 22 to 24, wherein the sample preparation module is further configured to retain an extraction agent for extracting the fatty acid esters from the fluid sample.

26. The microfluidic device of claim 25, wherein the extraction agent is an organic solvent.

27. The microfluidic device of any one of claims 22 to 26, wherein the sample preparation module comprises a blood filtration unit configured to separate whole blood into red blood cells (RBCs) and plasma.

28. The microfluidic device of any one of claims 22 to 27, wherein the sample preparation module comprises a normalization module configured to normalize a volume of the fluid sample.

29. The microfluidic device of any one of claims 22 to 28, wherein the sample preparation module comprises a reaction chamber configure to receive the fluid sample, the reaction chamber being fluidly coupled to the reservoir and configured to receive one or more of the organic solvent and the hydrolyzing agent from the reservoir to combine with the fluid sample.

30. The microfluidic device of any one of claims 22 to 28, wherein the reservoir is configured to receive the fluid sample to combine one or more of the organic solvent and the hydrolyzing agent with the fluid sample.

31. The microfluidic device of any one of claims 22 to 30, wherein the sample preparation module is configured to retain an agent configured to:
convert the free fatty acids to a fatty acid metabolite to be detected in a bioassay.

32. The microfluidic device of any one of claims 22 to 30, wherein the detection module is configured to retain an agent configured to:
convert the free fatty acids to a fatty acid metabolite to be detected in a bioassay.

33. The microfluidic device of any one of claims 22 to 32, wherein the free fatty acids include DHA and the DHA is converted to RvD1.

34. The microfluidic device of claim 33, wherein the DHA is converted to RvD1 by combining the DHA with an enzyme.

35. The microfluidic device of claim 34, wherein the enzyme is selected from a group consisting of: COX-1, 5-lipoxgenase (5-LOX), 12-lipoxgenase (12-LOX), 15-lipoxgenase (15-LOX), a combination of COX-2 and 5-LOX, cyclooxygenase-2 (COX-2), a combination of 15-LOX and 5-LOX, a combination of 12-LOX and 15-LOX and a combination of cyclooxygenase-2 (COX-2) and acetylsalicylic acid (ASA).

36. The microfluidic device of any one of claims 22 to 36, wherein the hydrolyzing agent is a chemical hydrolyzing agent.

37. The microfluidic device of claim 36, wherein the chemical hydrolyzing agent comprises one or more of the following: potassium hydroxide (KOH), sodium hydroxide (NaOH), deacylation with lithium hydroxide in chloroform-methanol, and/or hydrochloric acid (HCl).

38. The microfluidic device of any one of claims 22 to 36, wherein the hydrolyzing agent is an enzymatic hydrolyzing agent.

39. The microfluidic device of claim 38, wherein the enzymatic hydrolyzing agent is selected from a group consisting of: lipases, phospholipase, phospholipases A (PLA), phospholipase A2 (PLA2s), cytosolic phospholipases A2 (cPLA2s), iPLA2, Ca2+-independent phospholipase A2 (iPLA2 β), lipoprotein-associated phospholipases A2 (Ip-PLAs2), secreted phospholipases (sPLA2s), phospholipases D, Burkholderia cepacia (BC) lipase, Chromobacterium viscosum lipase, Lipase A Candida antarctica, Rhizopus oryzae (RO) lipase, pancreatin lipase, and a combination of two or more of any of the above.

40. The microfluidic device of claim 28, wherein the normalization module is a microchannel configured to normalize a volume of the fluid sample before the fluid sample enters the sample preparation module.

41. The microfluidic device of any one of claims 22 to 40, wherein the microfluidic device is a lateral flow device.

42. The microfluidic device of any one of claims 22 to 41, wherein a user adding the fluid sample to the microfluidic device may expect to receive an indicator of the quantity of the fatty acids or SPMs or fatty acid metabolites in about two hours or less.

43. The microfluidic device of any one of claims 22 to 42, wherein a period of time between a user adding the fluid sample to the microfluidic device and receiving an indicator of the quantity of the free fatty acids or SPMs or fatty acid metabolites from the microfluidic device is in a range of less than about 1 minute to about 30 minutes.

44. The microfluidic device of claim 43, wherein the period of time between the user adding the fluid sample to the microfluidic device and receiving the indicator of the quantity of the free fatty acids or SPMs or fatty acid metabolites from the microfluidic device is about 20 minutes.

45. The method of any one of claims 1 to 21, wherein the fatty acids are omega-3 fatty acids.

46. The method of any one of claims 1 to 21, wherein the fatty acids are omega-6 fatty acids.

47. The method of claim 1 or claim 2, wherein the fatty acid metabolite includes at least one of: lipoxins (Lx), resolvins (Rv), protectins (PD), neuroprotectins (NP), isofurans, isoprostanes, maresins (MaR), SPM intermediates and endocannabinoids.

48. The microfluidic device of any one of claims 22 to 44, wherein the fatty acids are omega-3 fatty acids.

49. The microfluidic device of any one of claims 22 to 44, wherein the fatty acids are omega-6 fatty acids.

50. The microfluidic device of claim 22, wherein the metabolite includes one or more of lipoxins (Lx), resolvins (Rv), protectins (PD), neuroprotectins (NP), isofurans, isoprostanes, SPM intermediates and maresins (MaR).

51. A method of quantifying a fatty acid metabolite present in a fluid sample on a microfluidic device, the method comprising:
performing a bioassay specific to the fatty acid metabolite on the microfluidic device.

52. A microfluidic device for quantifying a fatty acid metabolite in a fluid sample, the microfluidic device comprising:
a sample preparation module configured to receive the fluid sample; and
a detecting module fluidly coupled to the sample preparation module, the detecting module configured to receive the free fatty acids from the sample preparation module;
wherein one of the sample preparation module and the detecting module retains an assay agent to be bound to the fatty acid metabolite to quantify the fatty acid metabolite in the fluid sample.
